(19) United States
(12) Patent Application Publication
(10) Pub. No.: US 2003/0020875 A1
(43) Pub. Date: Jan. 30, 2003

(54) Spectral pattern ERG for detection of glaucoma

(76) Inventor: Harry G. Sperling, Northbrook, IL (US)

Correspondence Address: Wisner & Associates, Suite 930, 2925 Briarpark, Houston, TX, 77042, US

(21) Appl. No.: 10/166,473

(22) Filed: Jun. 10, 2002

Related U.S. Application Data
(60) Provisional application No. 60/296,981, filed on Jun. 8, 2001.

(51) Int. Cl. A61B 3/10; A61B 3/14

(52) U.S. Cl. 351/206; 351/211; 351/213

(57) ABSTRACT

Following from the finding that the spectral pattern ERG responds to both luminance and color, a test was devised that pits one against the other in such a way that a numeric score is derived which reflects the amount of color response required to cancel the luminance response. Contrary to expectations derived from the psychophysical literature on glaucoma, it was found that with the progress of glaucoma less and less color response is required to cancel luminance response indicating that the luminance ganglion cells deteriorate earliest. Tests on populations of normals, glaucoma suspects and confirmed glaucoma patients who do not yet show consistent visual field defects on the Humphrey Test, reveal significantly lower scores on this test for those with confirmed glaucoma than for the suspects and for both suspects and confirmed glaucomas than for the normals. There was no significant difference between test and retest, showing high reliability. It was also shown by tests on paralyzed and narcotized rhesus monkeys that earliest differences between normal control eye and eye with induced high intraocular pressure are detected outside the central 17 deg×10 deg in the retinal periphery with a 34 deg.×20 deg. checkerboard pattern. This was underscored by finding the largest difference between the response of treated and control eye with the larger pattern plus occlusion of the central 10 degrees. Finally, it was shown on two normal humans that larger pattern ERG response occurred to the larger of the two fields and were clearly discernable when the central 10 degrees were occluded. Therefore, it is concluded that a form of spectral pattern ERG visual field test is shown feasible in which recording responses to annular checkerboard patterns of different inner and outer diameters is contemplated. A test device for following the progress of glaucoma and the success of treatment at a time earlier in the course of the disease than can be revealed by the subjective visual field tests was also designed.

In accordance with the provisions of 35 U.S.C. 119(e), I hereby claim the benefit of the Jun. 8, 2001 filing date of application Serial No. 60/296,981.

The present invention relates to a method and apparatus for detecting glaucoma. In more detail, the present invention relates to the use of an apparatus for projecting a spectral pattern on the retina and recording the spectral pattern electroretinogram (SPERG) for early detection of glaucoma and monitoring of vision loss over time.

Comparisons of loss of ganglion cells with the visual field loss on human patients and experimental monkey preparations (Quigley, H. A., Dunkelberger, G. R., Green, W. R. Retinal ganglion cell atrophy correlated with automated perimetry in human eyes with glaucoma. Am J Ophthalmol. 1989; 107:453-464; Harwerth, R. S. et al. Ganglion cell losses underlying visual field defects from experimental glaucoma. Inves Ophthal Vis Sci 1999; 40:10:2242-2250) indicate that up to 50 percent of ganglion cells are already lost before the Humphrey Visual Field Test, the most widely used test for confirming the presence of glaucoma, begins to reliably show loss of vision. For this reason, it is generally agreed that a functional test for open angle glaucoma should be sought that shows changes from normal earlier in the course of the disease than the automated visual field tests. The evidence from which to guide the search, however, is contradictory. An important aspect of the findings on the relationship between visual field loss and loss of ganglion cells is the finding that in the mid-periphery, where glaucoma begins, there is selective earlier loss of the larger ganglion cells (Glovinsky, Y. et al. Retinal ganglion cell loss is size dependent in experimental glaucoma. Invest Ophthalmol Vis Sci 1991; 32: 484-491). It has been suggested that, because larger and smaller ganglion cells have been shown to serve different visual functions, a test of the functions served by the larger cells should be pursued as an efficient approach to a new test (Porciatti, V. et al. Responses to chromatic and luminance contrast in glaucoma: a psychopohysical and electrophysiological study. Vision Res 1997; 37: 1975-1987). The larger, parasol, ganglion cells synapse upon the cells of the magnocellular layers of the lateral geniculate nucleus (LGN) while the smaller, midget ganglion cells as well as the mid-sized blue-yellow ganglion cells, synapse on the cells of the parvocellular LGN layers. It is well established that magnocellular pathways serve brightness and temporal discrimination functions and the parvocellular serve color discriminations (Kaplan, E. and Shapley, R. M. The primate retina contains two types of ganglion cells, with high and low contrast sensitivity. Proc Natl Acad of Sci USA. 1986; 83: 2755-2757; Derrington, A. M. et al. Chromatic mechanisms in lateral geniculate nucleus of macaque. J Physiol 1984; 357: 241-265). Thus, tests selectively involving the former, perhaps tests of luminance discrimination combined with some temporally dependent aspect as distinct from those involving color, would be indicated. Yet other psychophysical data on glaucoma, both recent and dating back a number of years, show selective loss of blue-yellow discriminations earlier than red-green or luminance discriminations, implying early involvement of both magno- and parvocellular serving ganglion cells (Lakowski, R. and Drance, S. M. Acquired dyschromatopsias: the earliest functional losses in glaucoma. Documenta Ophthal Proc Ser, 19: 159-165; Sample, P. A. and Weinreb, R. N. Color perimetry for assessment of primary open angle glaucoma. Inves Ophthal Vis Sci 1990; 31:1869-1875; Porciatti, V. et al. (Op cit.)).

There is evidence that the Humphrey Visual Field test using blue on yellow stimuli (Sample, P. A. and Weinreb, R. N. Color perimetry for assessment of primary open angle glaucoma. Inves Ophthal Vis Sci 1990; 31:1869-1875) detects glaucoma earlier than the achromatic version. However, because the blue-yellow test is tediously slow to administer and patients, as reported by test administrators, dislike taking it, it must be judged as relatively inefficient. A number of discussions with patients have revealed that the major objection to all visual field tests, as a class, is the difficulty which most reported in deciding whether or not they saw the flashes. It is therefore an object of the present invention to provide a test for detection of glaucoma utilizing a spectral pattern electroretinogram (SPERG) that obviates that class of difficulty entirely. Specifically, it is an object of the present invention to provide a test for early diagnosis of glaucoma that only requires the patient to maintain fixation on a red dot in the center of the test pattern for a short time and to provide an electronic system that provides all other functions necessary to conduct the test.

Another relevant set of findings grew out of studies of the spectral sensitivity of the retina (Sperling, H. G. Visual colour processing is completed in the retina. In Colour Vision Deficiencies XIII. 1997;135-139)). These studies employed psychophysical increment threshold tests and electroretinographic (ERG) tests with different wavelengths of the visual spectrum in order to study the origins of color opponency in the different retinal layers. It was found that the photoreceptor and bipolar layers of the retina, as measured with ERG a- and b-wave responses to different wavelengths, largely showed only summation of red and green cone response while the pattern ERG showed large subtractive interactions between red and green response as well as large response to blue wavelengths. Because the pattern ERG disappears with ganglion cell degeneration without any effect on the a- or b-wave responses (Fiorentini, A. et al. The ERG response to alternating gratings in patients with diseases of the peripheral retinal pathways. Invest. Ophthal Vis Sci 1981; 21: 490-493; Maffei, L. and Fiorentini, A. Electroretinographic responses to alternating gratings before and after section of the optic nerve. Science 1981; 211: 953-955; Morrone, C. et al. Pattern-reversal electroretinogram in response to chromatic stimuli-II. Monkey. Vis Neurosci 1994;11: 861-871), color opponency and amplification of blue response largely originate in the ganglion cell layer. Comparison of pattern ERG spectral sensitivity with final common path psychophysical threshold spectral sensitivity also indicated that red-green opponency and blue amplification were completed in the retina at the ganglion cell level.

These studies resulted in a new pattern ERG technique, described in U.S. Pat. Nos. 5,382,987 and 5,506,633, both hereby incorporated into this specification in their entirety by this specific reference, that benefited from the stability of a null measurement. Using a reciprocating checkerboard pattern of spectral and white checks, white checks were held constant at a moderately high photopic luminance and the intensity of the spectral checks was varied in equal log steps from dimmer to brighter than the white checks. In doing so, it was found that the amplitude of the ERG went through a minimum, leading to the conclusion that at the step where the spectral and white stimuli were of equal luminance, there is minimum ERG response, akin to the disappearance of a black and white checkerboard pattern where adjacent checks are of equal luminance. Instead of zero ERG response, a minimum, but not zero, response was measured because of the color difference. But this result necessarily implied that this technique combines color and luminance responses, implying that both luminance and color opponent ganglion cells are activated by the pattern ERG stimulus. This finding is believed to relate to the functional-anatomical dichotomy between magnocellular and parvocellular pathways in the context of the earlier loss of larger ganglion cells in glaucoma and suggests the basis for a new functional test for early glaucoma.

Because it is possible to distinguish and measure the excitatory and inhibitory responses of each color receptor class and the way in which they combined in the responses of the ganglion cell layer of the retina (see the above-incorporated Sperling patents), and because glaucoma is a disease of degenerating ganglion cells, it seemed particularly impressive that, when the checkerboard pattern was defocused or replaced with successive large homogenous fields, which alternated on the same temporal schedule as the reciprocating checks, a spectral sensitivity curve was found that was identical with that of the bipolar cell layer measured with b-wave response to different wavelengths. It was therefore possible to go back and forth from the ganglion cell layer curve with a deep notch in the yellow from red-green inhibition and large blue peak to the smooth bipolar curve with no notch and no blue peak by introducing and removing the sharply focused borders of the reciprocating checkerboard.

It was therefore hypothesized that with selective loss of color response in glaucoma, predicted by others' color psychophysical studies, there would be a transition or degeneration from the former to the latter. It was, therefore, an object of the present invention to measure the spectral sensitivity of a pilot group of open angle glaucoma patients with the checkerboard test and compare the results with the results on normals. There were, however, considerable obstacles to doing so. Patients could not be expected to hold fixation in the Maxwellian view optical system with which the normal results were obtained on highly trained subjects and the need for intense blue light ruled out simple color monitors as stimulators. Further, even though a digital projection color monitor is available that provides ten times the blue intensity of ordinary color monitors with relaxed Newtonian viewing using rear projection on a high quality translucent screen, when experiments were conducted with a stimulus that was a rectangular checkerboard with 1cpd alternating spectral and white checks reciprocating at 16 times per second and viewing distance was one meter and the stimuli were limited to blue, green, red and yellow whose dominant wavelengths were 460 nm, 520 nm, 640 nm and 580 nm, obtaining adequate data on each wavelengths required at least four one hour sessions, placing an undue burden on the time of the patients. It was therefore another object of the invention to provide a method in which it is unnecessary to do so.

Another object of the present invention is to provide a method of assessing the efficacy of treatment for arresting the course of glaucoma.

This listing of several of the objects to which the invention is directed is provided merely to illustrate the rationale for the present invention and is not intended as a complete listing of all of the motivations for making the present invention.

### BRIEF DESCRIPTION OF THE DRAWINGS

FIG. 1 is a graph of the right eye scores of ten normal patients (in white), 14 patients suspected of glaucoma (in gray), and eighteen confirmed glaucoma patients (in black) as a function of age, the patients being scored on a scale of one through seven to denote increasing energies of blue (on a log 10 energy scale) as required to balance out the luminance response of a pattern ERG and cause it to disappear into noise, when tested in accordance in accordance with a preferred embodiment of the present invention.

FIGS. 2a-8g are graphs comparing the results with blue and green stimuli on a single monkey tested for glaucoma in accordance with a presently preferred embodiment of the method of the present invention.

FIGS. 3a and 3b are graphs comparing the results with blue and green stimuli for a second monkey tested for glaucoma in accordance with a second presently preferred embodiment of the method of the present invention.

FIG. 4 is a partially schematic view of a preferred embodiment of an apparatus for testing a patient for glaucoma constructed in accordance with the teachings of the present invention.

### DETAILED DESCRIPTION OF THE PREFERRED EMBODIMENT

It was surprising to find from preliminary measurements on glaucoma patients that, rather than requiring more of the blue stimulus to reach a minimum ERG amplitude against the constant white, as to be expected based on the psychophysical literature, patients required less blue to balance the white. This result has proven generally true and is evidence that in glaucoma the luminance ganglion cells deteriorate before the color opponent ganglion cells and meshes well with the finding that the larger ganglion cells are lost first. As a result, a test was configured based on a single waveband in the blue alternating with white. Most subjects were also tested with a second waveband in the green, which yielded very much the same results as the blue.

A further simplification was possible. For normal subjects, there was barely sufficient intensity of the blue light, even going to the highest blue intensity, to achieve more than several steps beyond the minimum. For over half of the glaucoma patients and many of the suspects, the pattern ERG disappeared below the noise level after a few intensity steps of the blue and either did not recover at higher blue intensities or did so erratically. Consequently, greater stability was achieved by scoring the intensity step of the blue stimulus at which the pattern ERG disappeared. In the case of the normals it never disappeared, so their score was 7, the highest intensity. The steps at which the patients' pattern ERG recovered beyond the step where it disappeared into the noise were ignored. This scoring procedure no doubt loses the ability to discriminate very slight losses versus normal response, but resulted in a considerable gain in overall stability. Therefore, the test is configured on an eight point scale, with zero denoting that no spectral light was required to balance the response to white because the pattern ERG response was always below the noise level. Scores of one through seven denote increasing energies of blue (on a log 10 energy scale) were required to balance out the luminance response in the pattern ERG and cause it to disappear into noise; so, the lower the score, the greater the visual deficit.

Thirty-two patients were studied, obtained from the practices of three ophthalmologists on the staff of The University of Texas—Hermann Eye Center in Houston. They fell in two categories: glaucoma suspects and confirmed glaucomas. All had prolonged high intraocular pressures (greater than 24 mm/Hg). Some were receiving medication that, at the time of testing, had brought pressure below that level. All had better than 20/30 acuity with correction in both eyes. They were chosen as having no visual field loss on the Humphrey, although it subsequently turned out that three of the confirmed glaucoma patients had minimum field loss. Those three subjects scored zero on the spectral pattern ERG test. The confirmed glaucoma diagnoses resulted from ophthalmoscopically observed changes in the optic disc.

Ten normal subjects, as dispersed in age as possible, were selected from among the staff of the Department. All had better than 20/30 acuity with correction and had had recent ophthalmological examinations. Ten of the patients were re-tested to evaluate test-retest reliability.

The subjects sat in an upright position in a dental chair which could be electrically raised and lowered and which had a finely adjustable headrest. Their eyes were one meter from a high quality rear projection screen, which was surrounded by a white equiluminant border whose extent was 30 eg. by 30 eg. The projection screen subtended 17 deg. horizontally by 10 deg. vertically. It was filled by the sharply focused image of the checkerboard projected from a Texas Instruments digital projection color monitor. The checks were alternately blue and white. The waveband of the blue stimulus was very close to square on energy coordinates and sharply cut off at 400 nm and 480 nm. The white was a combination of red, green and blue wavebands that plotted close to the CIE coordinates of 5000 deg. K. The white checks were held constant at 10,000 tds. The blue checks were varied in equal log 10 steps from at least four steps below the luminance of the white to at least four steps above the luminance of the white. The checks subtended 1 cycle/deg. They were alternated 16 times per second.

Thread electrodes of a type available commercially were placed under both lower eyelids and the affixed leads were connected to the input of a Nicolet ERG/EEG clinical amplifier and recording device, which was set to cumulate the voltage response of the retina (SPERG) to 250 alternations of the checkerboard (as triggered from the display generator). The Niclolet device was set to display the (time base averaged) responses to a sequence of four alternations of the checks at the end of the 250 programmed alternations. It also displayed and recorded, together with the four peak cumulative waveform, the mean peak-to-peak voltage of the SPERG. After each session, the operator scored the performance of each of the subject's eyes for the step on the intensity scale of the continuously varied spectral checks at which the four peaked patterns disappeared into the background noise.

On most subjects, data was recorded for green-white checkerboards of the same spatial, luminance and temporal characteristics as the blue. The results agree with the blue results in all regards and therefore are not set out here.

TABLE I

| | | SCORE | SCORE | SCORE | SCORE |
|---|---|---|---|---|---|
| GROUP | AGE | OD1 | OS1 | OD2 | OS2 |
| normal | 65 | 7 | 7 | | |
| normal | 60 | 7 | 6.8 | | |
| normal | 30 | 7 | 6.8 | | |
| normal | 29 | 7 | 6.8 | | |
| normal | 45 | 7 | 6.8 | | |
| normal | 41 | 7 | 6.8 | | |
| normal | 64 | 7 | 6.8 | | |
| normal | 66 | 7 | 6.8 | | |
| normal | 75 | 7 | 6.8 | | |
| normal | 30 | 7.2 | 6 | | |
| normal | 65 | 7 | 6 | | |
| suspect | 52 | 5 | 4.8 | | |
| suspect | 52 | 3 | | | |
| suspect | 55 | | 7 | | |
| suspect | 44 | 7 | 6.8 | | |
| suspect | 26 | 7 | 6.8 | 6 | 6 |
| suspect | 45 | 6 | 6.2 | | |
| suspect | 54 | 7 | 6 | | |
| suspect | 60 | 4.2 | 4.4 | 2 | 4 |
| suspect | 46 | 4 | 4.2 | | |
| suspect | 57 | 0 | 3 | 0 | 3 |
| suspect | 76 | 3 | 3 | | |
| suspect | 74 | 4 | 2 | | |
| suspect | 79 | 2.8 | 2 | | |
| suspect | 58 | 0 | 0.2 | | |
| suspect | 64 | 0 | 0.2 | | |
| suspect | 67 | 0 | 0.2 | | |
| glaucoma | 76 | 3 | 4 | 0 | 0 |
| glaucoma | 67 | 4 | 3.8 | 5 | 5 |
| glaucoma | 60 | 0 | 3.2 | 0 | 4 |
| glaucoma | 56 | 4 | 3 | | |
| glaucoma | 74 | 2.9 | 3 | | |
| glaucoma | 60 | 4 | 3.8 | | |
| glaucoma | 79 | 3 | 2 | | |
| glaucoma | 71 | 0.4 | 0.6 | | |
| glaucoma | 72 | 0 | 0.4 | | |
| glaucoma | 72 | 0.2 | 0.3 | 0 | 0 |
| glaucoma | 77 | 0 | 0.2 | 2 | 0 |
| glaucoma | 55 | 0 | 0.2 | | |
| glaucoma | 63 | 0 | 0.2 | 0 | 0 |
| glaucoma | 73 | 0 | 0.2 | | |
| glaucoma | 60 | 0.2 | 0 | 3 | 6 |
| glaucoma | 72 | 4 | 0 | | |
| glaucoma | 55 | 3 | | | |

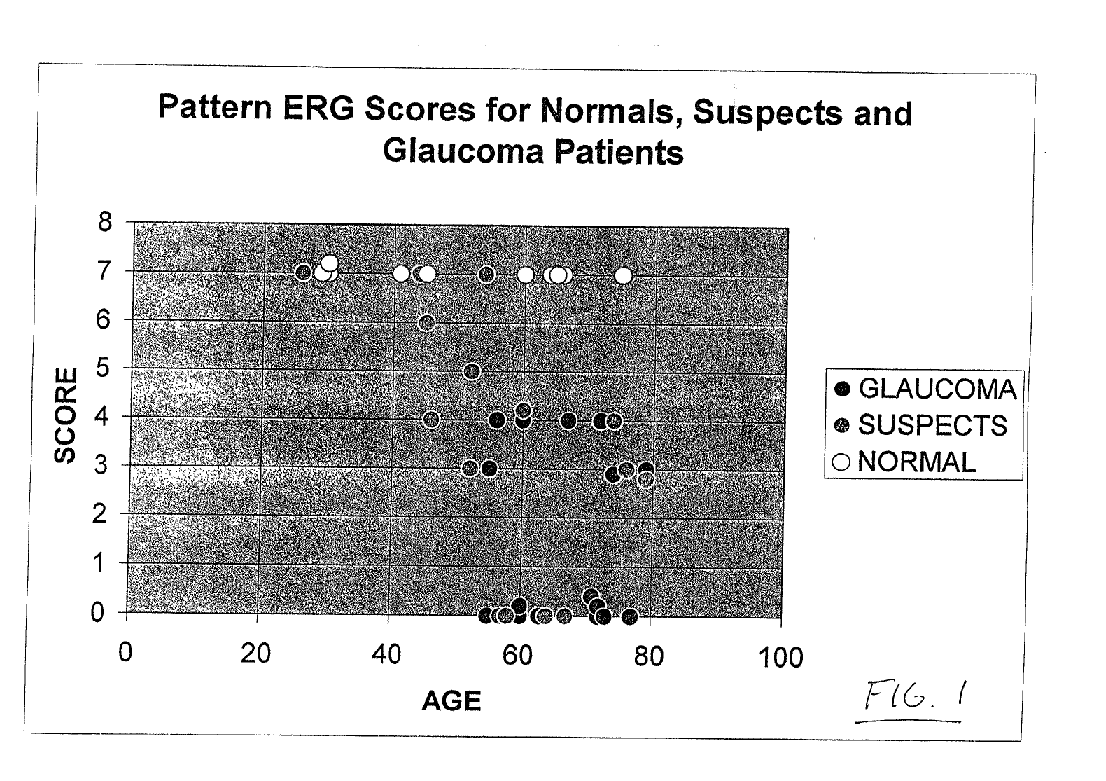

Table I shows the data. FIG. 1 shows a plot of the right eye scores of the 10 normals in white, of the 14 suspects in gray, and of the 18 confirmed glaucoma patients in black as a function of age. All ten normals scored 7, the top of the scale. The suspects' scores are evenly distributed over the entire range from 0 to 7 and tend to go down with age (to a statistically significant degree). Most striking and significant for evaluating the test, the scores of the confirmed glaucoma patients all fall in the bottom half of the scoring range, at or below 4. Thus the probability that the confirmed glaucoma patients' scores and the normals' scores were actually drawn from the same population is vanishingly small. In fact, the mean score for the suspects is also significantly different from that of the normals at better than the 0.01 level of confidence. There was no significant difference between the first and second testing of the ten repeated patients. The Pearson Product Moment correlation between first and second testing was r2=0.74. These results clearly indicate that the spectral pattern ERG test (SPERG) reliably distinguishes between eyes with and without primary open angle glaucoma at a stage of the disease earlier than can reliably be detected by automated visual field tests as exemplified by the Humphrey Visual Field Tester.

Thus, contrary to expectation, in glaucoma less spectral light, not more, is required to balance the white light input in the alternating spectral-white checkerboard stimulation. Therefore, the luminance ganglion cells must degenerate before the color-opponent ganglion cells in glaucoma. Further, the SPERG method of the present invention separates normal humans from glaucoma suspects from confirmed glaucoma patients with statistically different mean scores according to standard bio-statistical tests. It is also apparent that the SPERG method of the present invention shows significantly different scores between the normals, glaucoma suspects, and confirmed glaucoma patients) at a time when the standard functional test (the Humphrey Visual Field test using neutral flashes) does not yet show visual field loss.

Perhaps most significantly, the SPERG method of the present invention provides early detection automatically. In other words, unlike the Humphrey Visual Field test (which is, on information and belief, the standard test in the field), the method of the present invention does not require the patient to judge whether he/she sees the stimulus. The patient must only fixate on a red dot and the apparatus of the present invention (described below) provides all other functions for conducting the test. In a particularly preferred embodiment, the variability of the scores is reduced even further by excluding data where the patient's eyes have lost fixation.

The present invention also makes it possible to answer a question that could not previously be approached on human glaucoma patients, namely, how many ganglion cells have been lost without detection. In this study, high intraocular pressure was induced in one eye of a monkey by blocking trabecular mesh drainage with laser burn scars. Prior to treatment, fundus photographs and retinal nerve layer scans were obtained on each eye.

Because anesthetics greatly reduced the electrical response, great variability was encountered until tests were conducted on paralyzed and narcotized rhesus, which provided very large, stable pattern ERGs. This method provided high pressures on four monkeys and pattern ERG data, fundus photographs and nerve layer scans were obtained before and after induction of high pressure on each of their eyes. Two animals were sacrificed and their retinas preserved for histology.

All four monkeys showed a difference in the pattern ERG between the control eye and the treated eye. The pattern ERG had consistently lower amplitude in the treated eye at some time following treatment, providing good evidence that the spectral pattern ERG reveals substantial loss in the treated eyes, always the right eye (OD), versus the control eyes (OS), in all four animals.

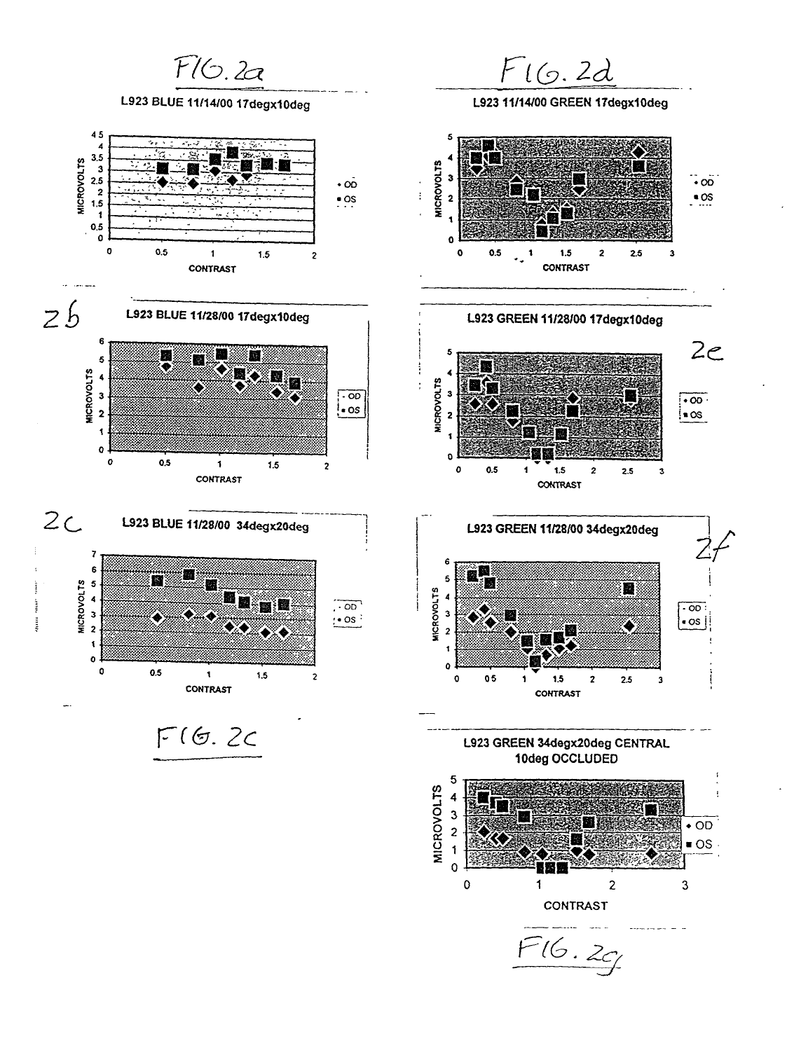

New data comparing central with peripheral vision obtained on two of the monkeys led to the conclusion that the method of the present invention is used to advantage to follow the progress of glaucoma beyond initial detection. On one of these animals (L923) spectral pattern ERGs were obtained with the same size 17 deg×10 deg field used on the human glaucoma patients and also with double that size field, 34 deg by 20 deg. In FIG. 2, a series of spectral pattern ERGs are shown for each eye of this monkey as a function of contrast. On the left in FIG. 2a, using a blue stimulus, it may be seen that about four weeks after the beginning of high pressure (approx 40 mm Hg) in the right eye, there is fairly consistently lower amplitude of the ERG in the right eye. In FIG. 2b, two weeks later with the same size field, the loss in the right eye is greater. In FIG. 2c, data are shown for the blue stimulus in a field that is twice as large (34 deg×20 deg). Here, the relative loss in the right eye versus the left eye is about three times as large, indicating that by far the largest loss lies beyond the smaller field in the retinal periphery. Further, using a green stimulus (FIGS. 2d through 2g), the green response is less sensitive to early loss than the blue, but the larger field shows loss that the smaller does not show, also ascribable to peripheral vs. central retinal degeneration. In FIG. 2g, with the green stimulus, data is shown taken with the 34 deg×20 deg field but with the central 10 degrees occluded is shown. Since the largest loss of amplitude in the right eye is shown for this condition, it is clear that the loss, as is well known for glaucoma, begins in the periphery.

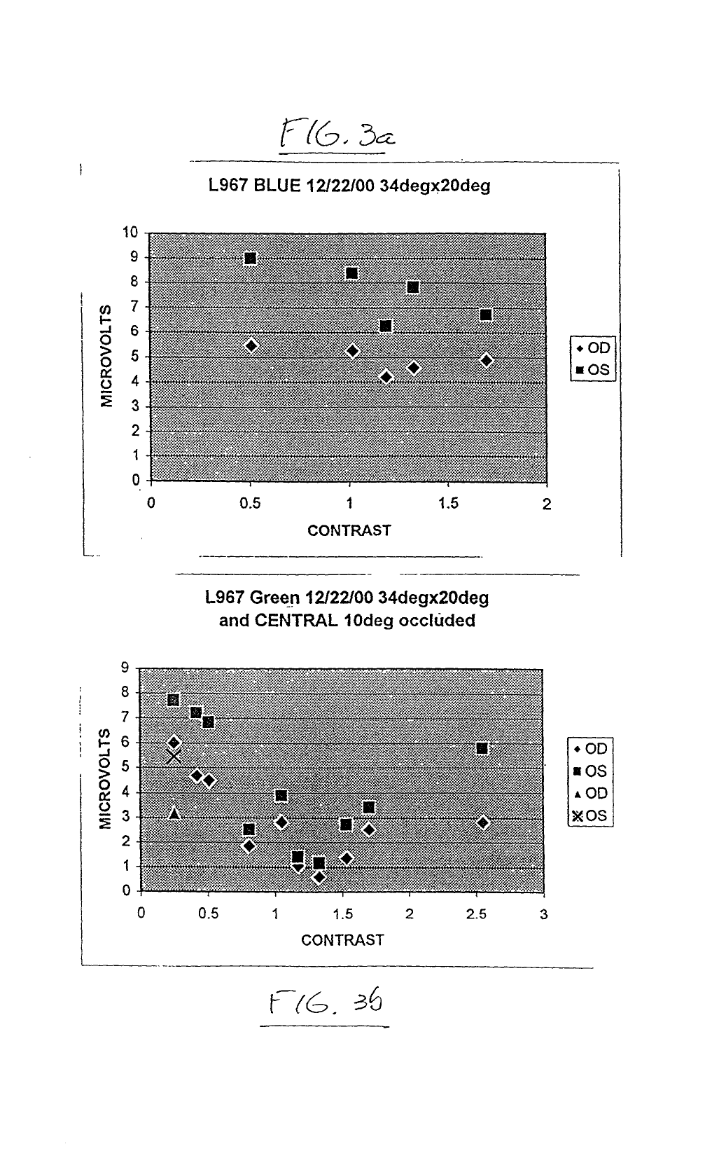

FIG. 3 shows data for a second monkey (L967), obtained on a single day after consistently elevated pressure readings had started in the treated eye. In FIG. 3a data are shown for the alternating blue and white checkerboard, showing substantially lowered sensitivity in the treated versus the normal eye. FIG. 3b shows that the green-white checkerboard yields a smaller difference, as had been shown for the other monkey. It also shows, for one contrast value, 0.25, data taken with the central 10 degrees of the field occluded (as the x for the normal eye and the triangle for the treated eye). Clearly, the peripheral field produced the larger difference, again supporting the conclusion from the first monkey that degeneration begins and is early detectable in the retinal periphery.

In a final test of the method and apparatus of the present invention, as learned from the data from the tests of patients cited above and the experiments on paralyzed and narcotized monkeys also cited above, two normal human subjects were tested with both the 17 deg by 10 deg field used on the patients and the larger 34 deg by 20 deg field from which data were obtained from the monkeys. The 34 deg. by 20 deg field yielded a larger pattern ERG amplitude than the 17 deg. by 10 deg. field, but more significantly, a substantial pattern ERG was obtained on these unanesthetized humans with the central 10 deg. occluded. This very significant result indicates that not only is the spectral pattern ERG (SPERG) an effective early detection test for glaucoma as learned from the study of glaucoma patients, suspects and normals cited above, such that all of the conditions are available to utilize SPERG as a visual field test for following the course of glaucoma after initial detection and before changes are detectable on Humphrey-type automated visual field tests. This is true because the course of glaucoma is from mid-peripheral towards central vision and the above-cited evidence establishes that before any changes are revealed by the Humphrey test, the method of the present invention detects early changes in the periphery. It was also shown that the present technique, and the test device described below, is capable of measuring peripheral visual response in awake humans, not just in paralyzed monkeys. This is an additional important application for the method of the present invention because it allows the clinician to follow the effectiveness of treatment at a time earlier than is possible with the Humphrey style subjective visual field tests.

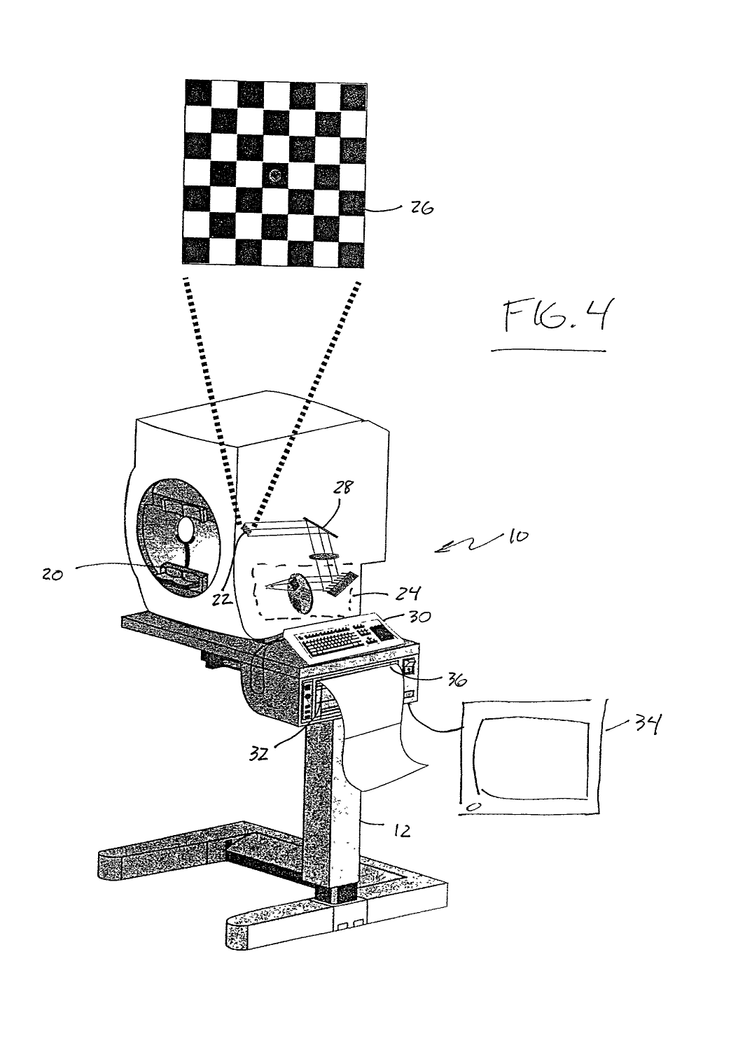

In the preferred embodiment of the apparatus of the present invention shown in FIG. 4, an apparatus 10 is provided comprising an adjustable table 12 having a hemisperical light adapting surround (not shown) which the patient sits in front of with the head in a chin and head rest 20. A substantially flat area 22 located at or near the center of the hemispherical surround is provided by a rear projector 24 and the test pattern 26 is projected via plane mirrors 28 from the projector 24. The tester operates the apparatus 10 from the keyboard 30 of a computer 32 that also serves to present, amplify, and process the SPERG. A monitor 34 and printer 36 may be provided to display and record the data. Also provided is software (not shown) that is stored in the memory of the computer for generating the display, or test pattern 26, and controlling projector 24, and for storing the results of the test to memory.

Those skilled in the art who have the benefit of this disclosure will recognize that certain changes can be made to the component parts of the present invention without changing the manner in which those parts function to achieve their intended result. For instance, the size of the fields described herein may be varied as known in the art while still functioning for the intended purpose. All such changes, and others which will no doubt be made clear to those skilled in the art by this description of the preferred embodiment, are intended to fall within the scope of the following, non-limiting claims.

What is claimed is:

1. A method of detecting glaucoma in a patient comprising the steps of:
projecting a checkerboard pattern electroretinogram onto the eye of a patient;
alternating the checks of the checkerboard pattern between white and colored checks of substantially constant intensity; and
increasing the intensity of the colored checks until the amplitude of the electrical response of the retina of the patient's eye to the alternating checks drops below a pre-selected threshold, as determined by the background noise level of the system, thus the pattern response disappears.

2. The method of claim 1 wherein the color of the checks is selected from the group consisting of blue and green.

3. The method of claim 1 additionally comprising assigning a score ranging from zero to seven indicative of the threshold, with zero denoting that no spectral light is required to balance the luminance response to white and a score of seven where the maximum available spectral light is unable to balance the luminance response.

4. The method of claim 3 wherein color intensity is increased in increments.

5. The method of claim 1 additionally comprising stimulating concentric spectral checkerboard patterns such as to measure relative response of different peripheral areas of the retina to test the progress of degeneration from the patient's peripheral towards the central field of vision.

6. The method of claim 5 for assessing the efficacy of treatment for arresting the course of glaucoma.

7. An apparatus for conducting the method of claim 1 by providing an alternating spectral-white checkerboard pattern and means for recording the response of a patient's eye to the pattern.
